(19) United States
(12) Patent Application Publication
(10) Pub. No.: US 2024/0352389 A1
(43) Pub. Date: Oct. 24, 2024

(54) FLUIDIC CHIP AND CELL ENCAPSULATION SYSTEM

(71) Applicant: TREEFROG THERAPEUTICS, PESSAC (FR)

(72) Inventors: Kevin ALESSANDRI, BOSTON, MA (US); Clément RIEU, BORDEAUX (FR)

(21) Appl. No.: 18/347,018

(22) Filed: Jul. 5, 2023

(30) Foreign Application Priority Data
Apr. 19, 2023 (FR) 2303923

(51) Int. Cl. C12M 3/06; C12M 1/12

(52) U.S. Cl. CPC C12M 23/16; C12M 25/16

(57) ABSTRACT

A fluidic chip composed of a body including a main inlet and at least one secondary inlet not coaxial to the main inlet, each main and secondary inlet being configured to receive a substance and an outlet, the main inlet is connected to the outlet by a main channel including a substantially rectilinear portion defining a central axis Z for the fluidic chip, the secondary inlet is also connected to the outlet by a secondary channel. The secondary channel is divided into at least two secondary ducts of the same dimension that join in a circular duct for injection coaxial with the main channel in an evenly distributed manner around the central axis Z.

### CROSS-REFERENCES TO RELATED APPLICATIONS

This application claims the benefit of the French patent application No. 2303923 filed on Apr. 19, 2023, the entire disclosures of which are incorporated herein by way of reference.

### FIELD OF THE INVENTION

The present invention relates to the technical field of fluidic chips and cell encapsulation systems comprising such a fluidic chip.

### BACKGROUND OF THE INVENTION

A fluidic chip is a set of channels etched or molded in a material such as glass, silicon or a polymer such as PDMS for PolyDiMethylSiloxane.

In the above domain, fluidic chips are known comprising inlets for receiving various substances and a single outlet through which the substances are thus combined. Fluidic chips comprise channels with sections whose dimensions are of the order of a millimeter to a micrometer which connect the inlets to the outlet.

Such fluidic chips can be manufactured by means of various techniques. The most common manufacturing technique comprises steps of molding the “PDMS” material or by embossing a thermoplastic material or by injection-molded thermoplastic or even also by means of a chemical lithography technique or a resin printing technique.

Most fluidic chips are used with liquid-liquid interfaces, between two liquids most often immiscible and have a two-dimensional architecture sufficient for this use. These interfaces can be used to make chemical reactions for example (microreactors) but are often used to form beads or capsules. When they use liquid-liquid interfaces, fluidic chips are only rarely used to form jets and most of the time, use dripping regimes. In order to form a stable jet in the air at the outlet of the chip, a 3D architecture is necessary. As for all fluidic chips, and even more so for their internal structures, great precision is necessary to avoid instabilities.

For the production of 3D or three-dimensional fluidic chips, the main technique is based on the printing of 3D photopolymerizable resins. Two types of two machines can then be used: widely marketed stereolithography printers such as “Formlab” or “Envisiontech” printers and femtosecond laser polymerization. Whereas the first technique makes it possible to produce objects several centimeters in length, the resolution only goes down to about fifty microns and does not make it possible to produce robust and reproducible structures less than a few hundred micrometers long. The second technique benefits from a very high spatial resolution of less than one micrometer but is not industrially usable for the production of parts comprising dimensions on the order of one centimeter. In addition, these techniques have the disadvantages of polymerizable resins which can make them unsuitable for their use in certain fields such as bio-production, or due to their chemical composition or even lack of certification.

A technique for the production of 3D fluidic chips is based on 3D-subtractive printing of glass (Selective Laser Etching) of molten silica, quartz, sapphire or borosilicate type. A laser thus emits the parts to be removed, to form channels in a glass plate after chemical visualization. The technique is intrinsically limited in terms of height due to the limited working distance of the optical system, all the more so since the precision to be achieved is large. The irradiated parts are then dissolved in an acidic or basic chemical bath. This method can last several days, hence the benefit of also limiting the length of the channels to avoid remaining in the bath too long. In addition to the impact on the productivity and speed of the process, excessively long bath times can create excessive geometric and dimensional alterations of the structures close to the inlets and outlets. In addition, according to this method, the cost of the part produced is proportional to the dimension along the central axis Z. Finally, the presence of long channels in the chips causes a greater pressure drop, which represents a real disadvantage in the use according to the application and the liquids used.

It is then necessary to find a way of compacting the dimensions along the central axis Z.

The method described above is used for manufacturing 3D fluidic chips for the production of a “shell-shell” type structure by double emulsion, such as “Raydrop” technology. Such chips comprise a 3D-printed glass head mounted on two coaxial capillaries thus enabling a drop-wise encapsulation in 3D. Limited by printing along the Z axis and the cost, only the head is 3D-printed, which therefore adds a complex mounting step to maintain a certain precision of coaxial capillaries. This type of structure is always in the liquid phase comprising immiscible liquids such as oil and water. The use of two coaxial capillaries represents a real fluid resistance which can represent a real limitation in its use, depending on the flow rates and fluids used.

Depending on the flow rates and the nature of the substances injected into each of the inlets of the fluidic chip, instability may appear in the various fluids flowing through the channels of the fluidic chip. Unlike dripping regimes, the production of jets requires greater flow rates which makes them more sensitive to instabilities, even more so for jets in the air rather than in another non-miscible liquid such as oil.

### SUMMARY OF THE INVENTION

The invention thus aims to overcome these various problems and proposes a simple-to-make fluidic chip for obtaining a coaxial jet at the outlet of the chip consisting of the various incoming substances. The invention makes it possible to compact the dimensions along the Z axis while ensuring a stable coaxial jet and without the need for a coaxial incoming injection of the fluids.

The invention therefore relates to a fluidic chip composed of a body comprising a main inlet and at least one secondary inlet not coaxial to the main inlet, each main and secondary inlet being intended to receive a substance and an outlet, the main inlet is connected to the outlet by a main channel comprising a substantially rectilinear portion defining a central axis Z for the fluidic chip, the secondary inlet is also connected to the outlet by a secondary channel.

According to the invention, the secondary channel is divided into at least two secondary ducts of the same dimension that join in a circular duct for injection coaxial with the main channel in an evenly distributed manner around the central axis Z. The implementation of secondary ducts of the same hydraulic resistance makes it possible to homogeneously divide the flow of substance injected into the corresponding secondary inlet while maintaining stability of the substance within and at the outlet of the chip. All the secondary inlets comprise secondary channels that divide into secondary ducts of the same dimension, meaning also the same cross-section and the same shape within the same secondary channel.

All the secondary ducts coming from the same secondary inlet join in a circular injection channel, coaxial to the main channel, in an evenly distributed manner relative to the main axis and to the same coordinate along the central axis Z in order to ensure homogeneous injection of the flows around the central axis.

The secondary injection ducts coming from the same secondary inlet join the main channel at the same coordinate along the central axis Z, or at different coordinates, allowing the creation of a flow composed coaxially of the various substances injected through the various main and secondary inlets. The creation of such coaxial jets makes it possible to obtain an isotropic flow according to the outlet XY dimensions and thus to avoid instabilities. The secondary injection channels coming from different secondary inlets do not necessarily join the main channel the same coordinate along the central axis Z.

At the outlet of the chip, any points of the flow that are symmetrical relative to the outlet axis, the properties of the stream are symmetrical relative to the outlet axis.

For any points of the symmetrical flow at the exit from the outlet axis, the minimum path between the points and their entry into the chip is substantially equal.

According to certain embodiments of the fluidic chip, a non-rectilinear portion not aligned with the central axis connects the main inlet to the main channel. This makes it possible to place the inlets along an axis different from the central axis Z.

According to one feature of the invention, the fluidic chip is obtained by a technique of subtractive 3D printing on glass (“Selective Laser Etching”). The glass used is of the molten silica, quartz, sapphire or borosilicate type, and preferentially made of molten silica. According to one embodiment, the fluidic chip is obtained by a subtractive 3D printing technique on a material allowing it. Such a material when it is removed from the material is capable of keeping great precision for the dimension of the channels while allowing the production of a part with a millimetric volume which can be greater than 1 mm3 and preferably greater than 10 mm3, 100 mm3 or even 1000 mm3.

According to one embodiment of the invention, the body of the fluidic chip comprises an upper face comprising the main inlet and at least the secondary inlet and a lower face opposite the upper face and comprising the outlet. The main inlet is thus aligned with the main channel and the outlet of the chip.

According to one feature of the invention, the geometry of the main and secondary channels is three-dimensional. Thus, the geometry cannot be complete in less than three two-dimensional planes.

According to another feature of the invention, the geometry of the fluidic chip is compact along the outlet axis. This means that the dimension of the set of channels along the outlet axis is not more than twice as much as the dimensions of all of the channels along the planes perpendicular to the outlet axis. It is thus possible to produce a fluidic chip whose dimension does not exceed 5 cm along the Z axis and according to other embodiments, the dimension along the Z axis of the fluidic chip may be 1 cm, or even lower.

According to one embodiment of the invention, the outlet axis coincides with the central axis Z.

According to one feature of the invention, the secondary channel comprises a first separation at an ordinate B of the central axis Z and a second separation at an ordinate D of the central axis Z, thus defining four secondary ducts of the same length. The implementation of a secondary channel comprising four secondary ducts of the same dimension and the same hydraulic resistance allows a maximum optimization of the occupancy of the height of the fluidic chip while ensuring a coaxial jet that is stable at the outlet.

According to another feature of the invention, the secondary channel comprises at least one rectilinear portion opening at the center of a portion in the shape of an arc of a circle extending in a plane perpendicular to the central axis Z. Using circular arc separations to form the secondary ducts makes it possible to obtain a tree architecture and therefore equivalent paths for the flow passing through the secondary channel. In addition, such symmetry relative to the central axis Z, a coaxial axis for the fluidic chip, makes it possible to increase the stability of the flows.

According to one embodiment of the invention, the fluidic chip comprises a first and a second secondary inlet connected respectively to a first and a second secondary channel, the first secondary channel comprising a portion in the shape of an arc of a circle of diameter D1 relative to the central axis Z extending in a plane perpendicular to the central axis Z, and the second secondary channel comprising a portion in the form of an arc of a circle of diameter D2 relative to the central axis Z extending in a plane perpendicular to the central axis Z, with the diameter D1 being less than the diameter D2.

Thus, the respective secondary ducts of each of the secondary inlets extend along circular arcs of different diameters. This architecture in particular has the advantage of not crossing the secondary ducts while retaining the equivalent path principle for each secondary channel. The respective secondary ducts therefore have different dimensions depending on the corresponding secondary channel but for the same secondary channel, each of the secondary ducts corresponding to the same dimension.

According to a feature of the invention, the first and second secondary channels each comprise two circular arc-shaped portions at two different ordinates from the central axis Z thus each defining four secondary ducts of the same size. The separations are thus carried out on the same ordinate, which facilitates the manufacture of the fluidic chip.

According to one embodiment of the invention, the main channel comprises four main ducts of the same size. The implementation of four main ducts in particular makes it possible to stabilize the flow passing through the main channel.

According to another feature of the invention, each main and secondary channel comprises a circle-shaped portion opening onto the outlet, each circle-shaped portion being concentric. This architecture has the advantage of creating coaxial jets at the outlet of the fluidic chip, which promotes certain methods of using the fluidic chip requiring concentric fluids.

The invention also relates to a system for encapsulating cells, the system comprising at least two containers, one of the containers comprising a solution of cells and the other of the containers comprising a solution capable of gelling, an encapsulation device comprising a fluidic chip according to the invention, the main and secondary inlets each being connected to one of the containers via a dispenser and able to form a concentric jet from the solutions provided by the dispensers, the encapsulation device being arranged so that the jet is divided, at the outlet of the encapsulation device, into drops whose exterior layer is the solution capable of gelling and the core of the solution of cells, and a gelling bath arranged downstream of the encapsulation device to collect the drops formed by the encapsulation device and arranged to cause a gelling of the exterior layer of each drop during its immersion in the bath.

The use of such a fluidic chip promotes the high-throughput cell encapsulation system (compared to drop-by-drop), in particular thanks to the creation of a concentric jet at the outlet of the fluidic chip according to the invention. The fluidic chip according to the invention can also be used for a drop-by-drop outlet.

Of course, the various features, variants and embodiments of the invention can be associated with one another according to various combinations insofar as they are not incompatible or exclusive of one another.

### BRIEF DESCRIPTION OF THE DRAWINGS

In addition, various other features of the invention will become apparent from the appended description given with reference to the drawings which illustrate non-limiting forms of embodiment of the invention and wherein:

FIG. 1 is a perspective view of an embodiment of a fluidic chip according to the invention whose inner channels are visible by transparency of the body of the fluidic chip,

FIG. 2 is a perspective view of the fluidic chip of FIG. 1 comprising a different viewing angle,

FIG. 3 is a half-section view of the fluidic chip of FIG. 1 at A different viewing angle at the cutting plane passing through the ordinate A of the central axis Z,

FIG. 4 is a half-section view of the fluidic chip of FIG. 1 at a different viewing angle at the cutting plane passing through the ordinate B of the central axis Z,

FIG. 5 is a half-section view of the fluidic chip of FIG. 1 at a different viewing angle at the cutting plane passing through the ordinate C of the central axis Z,

FIG. 6 is a half-section view of the fluidic chip of FIG. 1 at a different viewing angle at the cutting plane passing through the ordinate D of the central axis Z,

FIG. 7 is a half-section view of the fluidic chip of FIG. 1 at a different viewing angle at the cutting plane passing through the ordinate E of the central axis Z,

FIG. 8 is a half-section view of the fluidic chip of FIG. 1 at a different viewing angle at the cutting plane passing through the ordinate F of the central axis Z, and

FIG. 9 is a half-section view of the fluidic chip of FIG. 1 at a different viewing angle at the cutting plane passing through the ordinate G of the central axis Z.

### DETAILED DESCRIPTION OF THE PREFERRED EMBODIMENTS

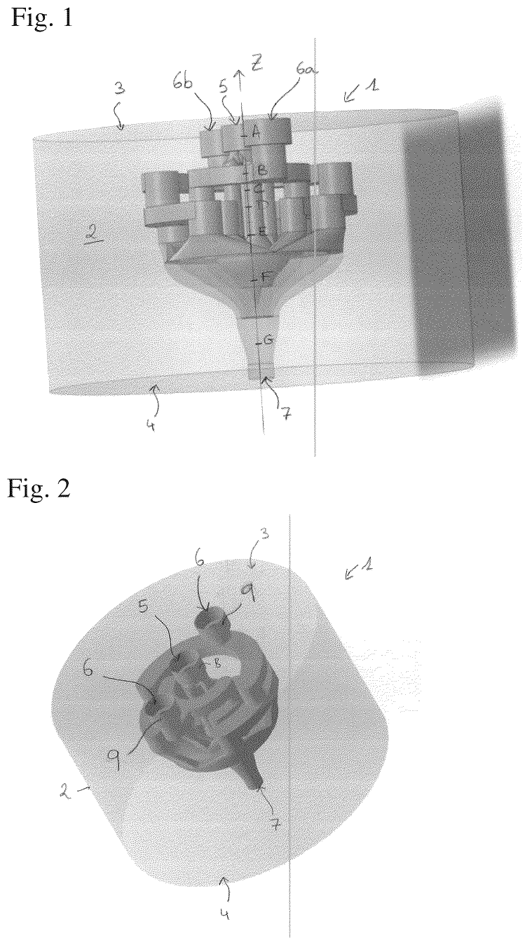

The dashed line in FIG. 1 corresponds to the central axis Z of the fluidic chip.

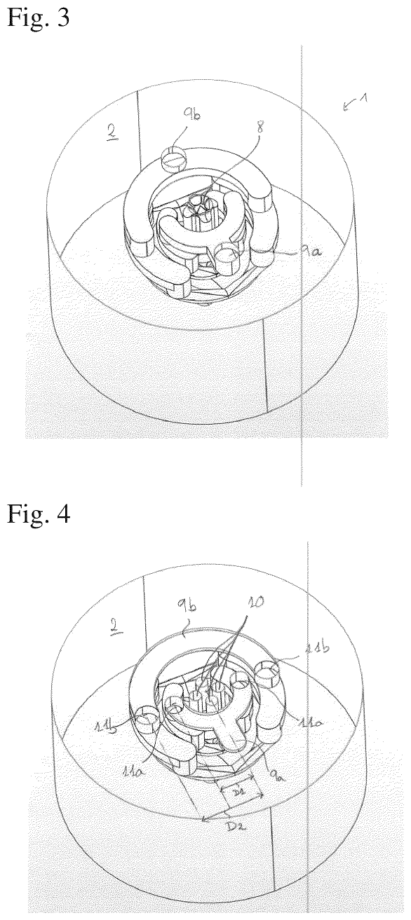

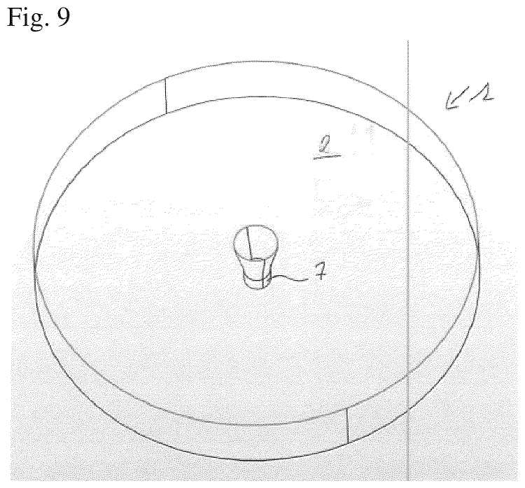

The bold lines of FIGS. 3 to 9 represent the cutting planes at each of the ordinates denoted by A to G on the central axis Z of FIG. 1.

It should be noted that in these figures, the structural and/or functional elements common to the different variants may have the same references.

The invention aims to stabilize the jet at the outlet of a fluidic chip.

For these purposes, the invention denoted by the reference 1 in its entirety a fluidic chip consists of a body 2 comprising an upper face 3 and opposite a lower face 4. The upper face 3 comprises a main inlet 5 and at least one secondary inlet 6. The lower face 4 comprises an outlet 7.

The figures show a fluidic chip 1 comprising a body 2 of circular cylindrical shape about a central axis Z with an upper face 3, a lower face 4 and a lateral face. Other embodiments such as polygonal are compatible with the invention. Thus, the faces of the body 2 of the fluidic chip 1 are not necessarily opposite.

A main channel 8 connects the main inlet 5 to the outlet 7. The main channel 8 is substantially rectilinear. The main inlet 5 is thus aligned on the outlet 7 along the central axis Z shown in FIG. 1. According to the embodiment shown, the main channel 8 substantially follows the central axis Z of the fluidic chip 1.

A fluidic chip 1 according to the invention also comprises a secondary channel 9 that connects the secondary inlet 6 to the outlet 7. A fluidic chip 1 according to the invention may comprise several secondary inlets 6, the fluidic chip 1 then comprises an equal number of secondary channels 9 which respectively connect each secondary inlet 6 to the outlet 7.

According to the embodiment shown, the main channel 8 and the secondary channel(s) 9 comprise partially circular sections whose dimensions vary and also sections of more complex shape, for example at the separations of a channel in two parts named ducts and described below.

FIGS. 1 and 2 show an embodiment of a fluidic chip 1 comprising a first secondary inlet 6a and a second secondary inlet 6b arranged on either side of the main inlet 5, the three main inlets 5 and secondary 6a, 6b being aligned. According to other embodiments of the invention not shown, a fluidic chip 1 comprises a single secondary inlet 6 or three or more secondary inlets 6. The architecture principle of the secondary channels 9 that are articulated around the main channel 8 is identical and is explained below. According to other embodiments not shown, the secondary inlets 6 may not be aligned.

FIG. 3 corresponds to a cross-section of FIG. 1 at the ordinate denoted A on the central axis Z in FIG. 1. The main inlet 5 is thus connected to the main duct 8. The first secondary inlet 6a is connected to the first secondary duct 9a and the second secondary inlet 6b is connected to the second secondary duct 9b.

FIG. 4 corresponds to a cross-section of FIG. 1 at the ordinate denoted B on the central axis Z in FIG. 1. The main channel 8 is divided by forming four rectilinear main ducts 10 parallel to each other. Furthermore, the secondary channels 9a, 9b are each divided, forming two secondary ducts 11a, 11b. The first secondary channel 9a comprises a rectilinear portion so as to come closer to the main channel 8 and then comprises a separation forming an arc of a circle, of diameter D1 relative to the central axis Z. The second secondary channel 9b comprises a separation forming an arc of a circle, of diameter D2 relative to the central axis Z. Thus D1 is less than D2 so as to optimize the space included in the plane and in particular situated on the ordinate denoted B.

According to one embodiment not shown, the fluidic chip 1 comprises a third secondary inlet 6 and therefore a third secondary channel 9. Said third secondary channel 9 then comprises a separation forming an arc of a circle of diameter D3 greater than the diameter D2 of the arc of a circle coming from the second secondary channel 9. Depending on the arrangement of the third secondary inlet, the third secondary channel 9 comprises a rectilinear portion making it possible to place the arc of a circle corresponds to the desired diameter D3. This architecture comprising concentric circular arcs is arranged according to the number of secondary inlets 6.

Advantageously according to the embodiment shown, the separation of each secondary channel 9 takes place at the middle of the arc of a circle in order to form secondary ducts 11 of the same length.

A secondary channel 9 of a fluidic chip 1 according to the invention therefore comprises at least one rectilinear channel portion substantially parallel to the central axis Z.

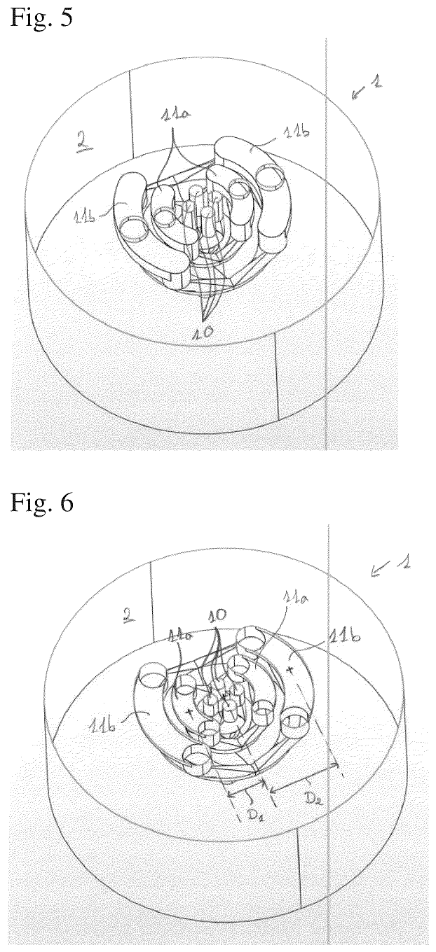

The four ducts 11a, 11b are also visible in FIG. 5 which corresponds to a section in FIG. 1 at the ordinate denoted C on the central axis Z in FIG. 1.

According to the embodiment shown, the secondary channels 6a, 6b comprise a second division visible in particular in FIG. 6 which corresponds to a section in FIG. 1 at the ordinate denoted D on the central axis Z in FIG. 1. The secondary channels 6a, 6b therefore split up twice, forming four secondary ducts 11a, 11b. This second separation also comprises portions in the shape of an arc of a circle around the central axis Z of the fluidic chip 1 according to the same diameters D1, D2 as for the first separation. The respective circular arcs of the first and second secondary channels 9a, 9b are symmetrical.

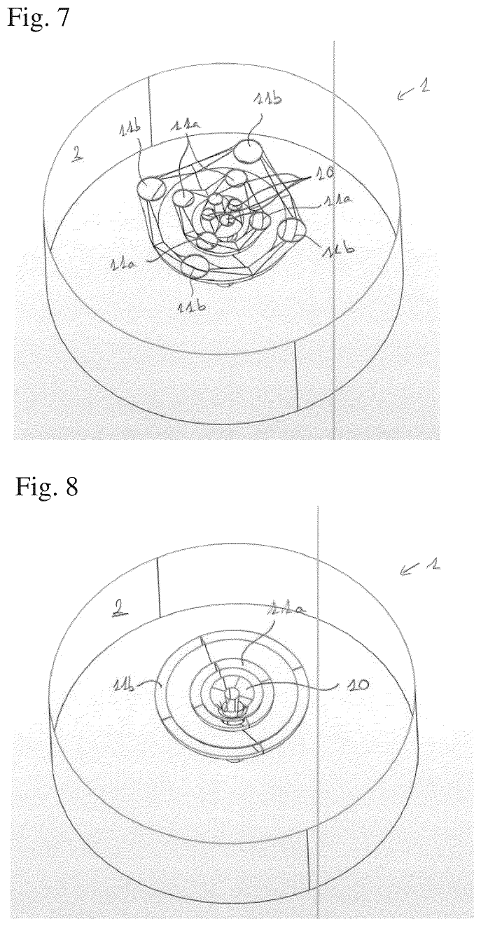

The four secondary ducts 11a, 11b thus formed from each of the secondary channels 9a, 9b are visible in FIG. 7 which corresponds to a section in FIG. 1 at the ordinate denoted E on the central axis Z in FIG. 1. Twelve main 10 and secondary ducts 11a, 11b are thus visible.

All the main 8 and secondary 9 channels meet at the outlet 7 on a coaxial circular injection duct. According to the embodiment shown and in particular in FIG. 8, which corresponds to a section in FIG. 1 at the ordinate denoted F on the central axis Z in FIG. 1, each main 10 and secondary 11a, 11b duct meets respectively on a respective single duct of circular shape. Each main 10 and secondary duct 11a, 11b being concentric so that all the ducts join together concentrically towards the outlet 7 in the coaxial circular injection duct. Thus, all the jets are coaxial at the outlet of the fluidic chip 1. According to the embodiment shown, the outlet 7 emerges directly into the air. According to other embodiments not shown, the outlet 7 is connected to an accessory or extension such as a nozzle.

According to one form of use, a fluidic chip 1 is used for a cell encapsulation system.

The cell encapsulation system comprises at least two containers, one of the containers comprising a cell solution and the other of the containers comprising a solution capable of gelling.

The cell encapsulation system also comprises an encapsulation device comprising a fluidic chip 1 according to the invention, the main 5 and secondary 6 inlets each being connected to one of the containers via a dispenser. The fluidic chip 1 is able to form a concentric jet at the outlet from the solutions provided by the dispensers. The encapsulation device is arranged so that the jet splits, at the outlet of the encapsulation device, into drops whose outer layer is the solution capable of gelling and whose core is the cell solution.

Finally, the cell encapsulation system comprises a gelling bath arranged downstream of the encapsulation device to collect the drops formed by the cell encapsulation device and arranged to cause a gelling of the outer layer of each drop during its immersion in the bath.

Of course, various other modifications can be made to the invention within the scope of the appended claims.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1-12. (canceled)

13. A fluidic chip composed of a body comprising:
a main inlet and
at least one secondary inlet not coaxial to the main inlet,
each main and secondary inlet being configured to receive a substance and an outlet,
the main inlet being connected to the outlet by a main channel comprising a substantially rectilinear portion defining a central axis Z for the fluidic chip,
the secondary inlet also being connected to the outlet by a secondary channel,
wherein the secondary channel is divided into at least two secondary ducts having identical dimensions which join in a coaxial circular injection duct with the main channel in an evenly distributed manner around the central axis Z.

14. The fluidic chip according to claim 13 obtained by a subtractive printing technique on glass.

15. The fluidic chip according to claim 13, wherein the body comprises an upper face comprising the main inlet and at least the secondary inlet and a lower face opposite the upper face and comprising the outlet.

16. The fluidic chip according to claim 13, wherein the geometry of the main and secondary channels is three-dimensional.

17. The fluidic chip according to claim 13, wherein the geometry of the fluidic chip is compact along the outlet axis.

18. The fluidic chip according to claim 13 wherein the secondary channel comprises a first separation on an ordinate B of the central axis Z and a second separation at an ordinate D of the central axis Z, thus defining four secondary ducts of the same length.

19. The fluidic chip according to claim 13, wherein the secondary channel comprises at least one rectilinear portion opening at a center of a portion in the shape of an arc of a circle extending in a plane perpendicular to the central axis Z.

20. The fluidic chip according to claim 13, comprising a first and a second secondary inlet connected respectively to a first and a second secondary channel, the first secondary channel comprising a portion having a shape of an arc of a circle of a first diameter relative to a central axis extending in a plane perpendicular to the central axis, and the second secondary channel comprising a portion having a shape of an arc of a circle of a second diameter relative to the central axis extending in a plane perpendicular to the central axis, with the first diameter being smaller than the second diameter.

21. The fluidic chip according to claim 20, wherein the first and the second secondary channels each comprise two circular arc-shaped portions at two different ordinates from the central axis Z, thus each defining four secondary ducts having a same length.

22. The fluidic chip according to claim 13, wherein the main channel comprises four main ducts having a same length.

23. The fluidic chip according to claim 13, wherein each main and secondary channel comprises a circle-shaped portion opening onto the outlet, each circle-shaped portion being concentric.

24. A cell encapsulation system, the system comprising:
two containers, one of the containers comprising a cell solution and the other of the containers comprising a solution capable of gelling,
an encapsulation device comprising a fluidic chip according to claim 13,
the main and secondary inlets each being connected to one of the containers via a dispenser and able to form a concentric jet from the solutions provided by the dispensers, the encapsulation device being arranged so that the jet splits, at the outlet of the encapsulation device, into drops whose outer layer is the solution capable of gelling and whose core is the cell solution, and
a gelling bath arranged downstream of the encapsulation device to collect the drops formed by the encapsulation device and arranged to cause a gelling of the outer layer of each drop during its immersion in the bath.
